(19) United States
(12) Patent Application Publication
(10) Pub. No.: US 2005/0193477 A1
(43) Pub. Date: Sep. 8, 2005

(54) Protective headgear

(76) Inventor: Martin Penny, Whitestone, NY (US)

Correspondence Address: F. CHAU & ASSOCIATES, LLC, 130 WOODBURY ROAD, WOODBURY, NY, 11797, US

(21) Appl. No.: 10/979,748

(22) Filed: Nov. 1, 2004

Related U.S. Application Data
(63) Continuation-in-part of application No. 10/212,539, filed on Aug. 5, 2002, now abandoned.
Application No. 10/212,539 is a continuation of application No. 09/814,310, filed on Mar. 21, 2001, now Pat. No. 6,427,253.

(51) Int. Cl. A61F 9/00

(52) U.S. Cl. 2/411; 2/425

(57) ABSTRACT

A protective headgear comprising a headband and two shield plates. In a back region, the headband and the shield plate extend down behind the user's ear towards a bottom region on a user's head. The shield plate has a bottom protective rim that is designed to cup underneath a user's skull to shield the base of the user's skull. In addition, both shield plates have a small gap between them so that they wrap around the user's head. Additional components can also be added to this headgear. For example, this headgear can also include a terry cloth in a front region to gather sweat or moisture from a user's forehead. In addition, two ear flaps can also be attached to the headgear to cover a user's ears. These earflaps are designed to keep the user warm in the winter. In addition, this headgear can be alternately opened and closed via a releasable factor such as a hook and loop fastener.

### CROSS-REFERENCE TO RELATED APPLICATIONS

This application is a Continuation-in-Part of U.S. patent application Ser. No. 10/212,539, filed on Aug. 5, 2002, which is a Continuation of U.S. patent application Ser. No. 09/814,310, filed on Mar. 21, 2001, now U.S. Pat. No. 6,427,253, all of which are fully incorporated herein by reference.

### SUMMARY OF THE INVENTION

Exemplary embodiments of the invention generally include removable protective headgear devices that include an integrally formed headband and having protective support plates attached thereto, which provide protection for the back of a user's head. Exemplary protective headgear devices according to the invention could be used for protection when roller skating, bicycling, snowboarding, rollerblading or any other activity that would require additional head protection.

More specifically, exemplary embodiments of the invention include protective headgear devices in the form of a headband having semi-rigid or rigid protective support plates/shields that can be attached to or detached from the headgear. The support plates can be formed of plastic or a substantially rigid rubber composite. In other embodiments, the support plates can be mounted to the headband either as an insert or as an outer attachment. For example, if the plates are applied as an insert, the plates are designed to insert between two sheets of the headband and then be fastened in via a top flap. Thee support plates can also be fastened to the back of the headband via a hook and loop fastener, a tongue and groove connection, glue, or sewn on for a more permanent attachment or any other fastening means known in the art.

Both the headband and the plates are shaped so that the headgear extends above both of the user's ears so that in warmer months, the user can hear any surrounding noise. The user can also attach ear pieces to the headband so that in colder months, the headband covers the user's ears.

The headband may be formed of a flexible elastic material such as neoprene rubber, or fleece with an elastic headband attached inside. This headband could also be covered with a hook and loop fastener friendly material comprising a mesh of fine loops. In addition, the headband can be opened by detaching a hook and loop fastener strap which allows the headband to be opened and removed from a person's head. The headband can be used for both warmth and protection of the individual.

These and other exemplary embodiments, aspects, features and advantages of the present invention will be described or become apparent from the following detailed description of exemplary embodiments, which is to be read in connection with the accompanying drawings.

### BRIEF DESCRIPTION OF THE DRAWINGS

FIG. 1 is a schematic perspective view of a protective headgear device according to an exemplary embodiment of the invention.

FIG. 2 is a schematic perspective view of a protective headgear device according to another exemplary embodiment of the invention.

FIG. 3 is a schematic perspective view of a protective headgear device according to another exemplary embodiment of the invention.

FIG. 4 is a schematic perspective view of a protective headgear device according to another exemplary embodiment of the invention.

FIGS. 5A and 5B are schematic back and top views of support plates according to an exemplary embodiment of the invention.

FIGS. 6A and 6B are schematic side views of support plates according to an exemplary embodiment of the invention.

FIGS. 7 and 8 are schematic perspective views of a protective headgear device according to another exemplary embodiment of the invention.

FIG. 9 is a schematic perspective view of a protective headgear device according to another exemplary embodiment of the invention.

FIGS. 10 and 11 are schematic perspective views of a protective headgear device according to another exemplary embodiment of the invention.

FIG. 12 is a schematic perspective view of a protective headgear device according to another exemplary embodiment of the invention.

### DETAILED DESCRIPTION OF EXEMPLARY EMBODIMENTS

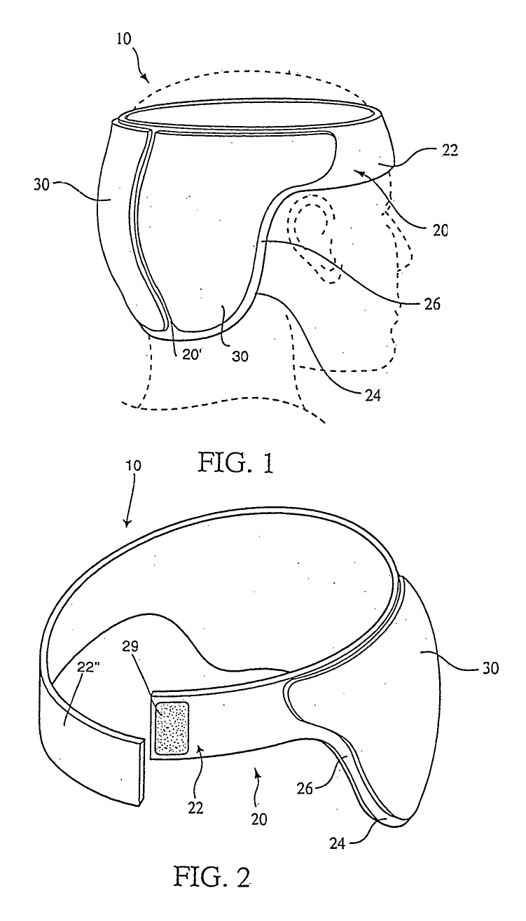

FIG. 1 is a schematic perspective view of a protective headgear device according to an exemplary embodiment of the invention. In particular, FIG. 1 illustrates a headgear device 10 comprising integrally formed headband 20 device and a plurality of support plates 30 (or impact shields) that are permanently or removably attached to a back region of the headband 20. The headband (20) can be formed from any suitable flexible, elastic material such as synthetic rubber materials (e.g., neoprene), foam rubber materials or other types of materials that are designed to act flexibly and durably, and which are preferably resistive to breakdown by water. In other embodiments, the headband 20 may be formed of fleece material with an elastic headband attached inside. The headband 20 comprises a front section 22 and a back section 24. Connecting the front section 22 and back section 24 is an angled/tapered mid section 26. Front section 22 is designed to wrap around a user's forehead while still allowing the user visibility. The front section 22 is designed to have a smaller cross sectional area than the back section 24.

Supports 30 can be designed as either semi-rigid or substantially rigid plates depending on the type of protection desired by the user. The supports 30 are designed to effectively disperse/spread impact energy. The back section 24 is designed to hold supports 30 either permanently or in a removable manner with a small gap 20′ between both supports 30. Back section 24 has a larger surface area than front section 22 and substantially covers a back region on a user's head. With this design, back section 24 provides coverage and protection for a back region of a user's head.

The angled/tapered mid section 26 is designed to connect front section 22 with back section 24. When headband 20 is placed on a user's head, angled mid section 26 slopes down between front section 22 and back section 24 wherein angled mid section 26 is angled above a user's ears to allow a user to hear the surrounding noise.

FIG. 2 is a schematic perspective view of a protective headgear device according to another exemplary embodiment of the invention. In particular, the exemplary embodiment of FIG. 2 is similar to the exemplary embodiment of FIG. 1, but further comprises an integrally formed headband 20 device that is designed as an open headband having a fastener 29 such as a hook and loop fastener for attaching two portions 22′ and 22″ together in front section 22. The fastener 29 may comprise adjustable velcro straps for custom fit. With this design, front region 22 can be alternately opened and closed around a user's head so that headband 20 can be easily wrapped around a user's head.

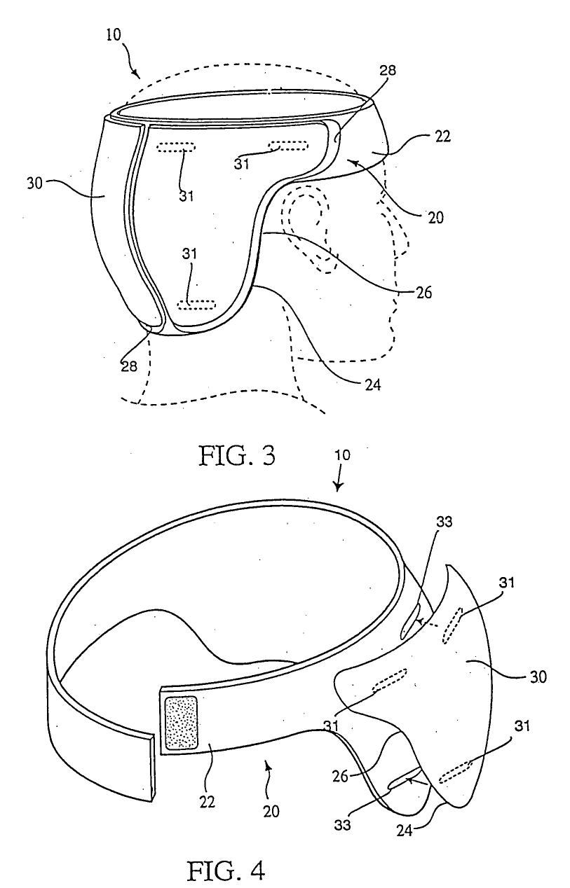

FIG. 3 is a schematic perspective view of a protective headgear device according to another exemplary embodiment of the invention. In this exemplary embodiment, an additional headpiece 28 is added, wherein the headpiece 28 is shaped similar to supports 30 and formed from the same or similar material of that of the headband 20. In other exemplary embodiments, the headpiece may be formed of any suitable impact-absorbing foam material known in the art. Headpiece 28 is disposed underneath supports 30 and provides additional padding for the user. In addition, FIG. 3 also shows supports 30 that can be attached to, or removed from headband 20 via fasteners 31. Fasteners 31 are disposed on a top region and on a bottom region on supports 30 so that they fully secure supports 30 to headband 20. As shown in FIG. 4, fasteners 31 on supports 30 fasten to fasteners 33 disposed on headband 20 wherein fasteners 31 and 33 can combine to form a hook and loop fastener, snaps, a tongue and groove connection, or any other type fastening means known in the art. Thus these fasteners allow supports to be alternately attached to headband 20 or detached from headband 20 so that headband 20 can be washed without damaging supports 30.

Supports 30 attach to an outer face of headband 20 primarily in back region 24. Supports 30 can be designed from a semi-rigid plastic, composite or metal-based material that is sufficiently rigid to displace energy related to a sharp contact or blow to the user's head resulting from a fall. Supports 30 are placed on the headband 20 so that when a user's head contacts an object resulting from a fall, the pressure or energy created from impact affects a substantially larger portion of the user's head but with a much lower force per surface area with the result probably being less injury.

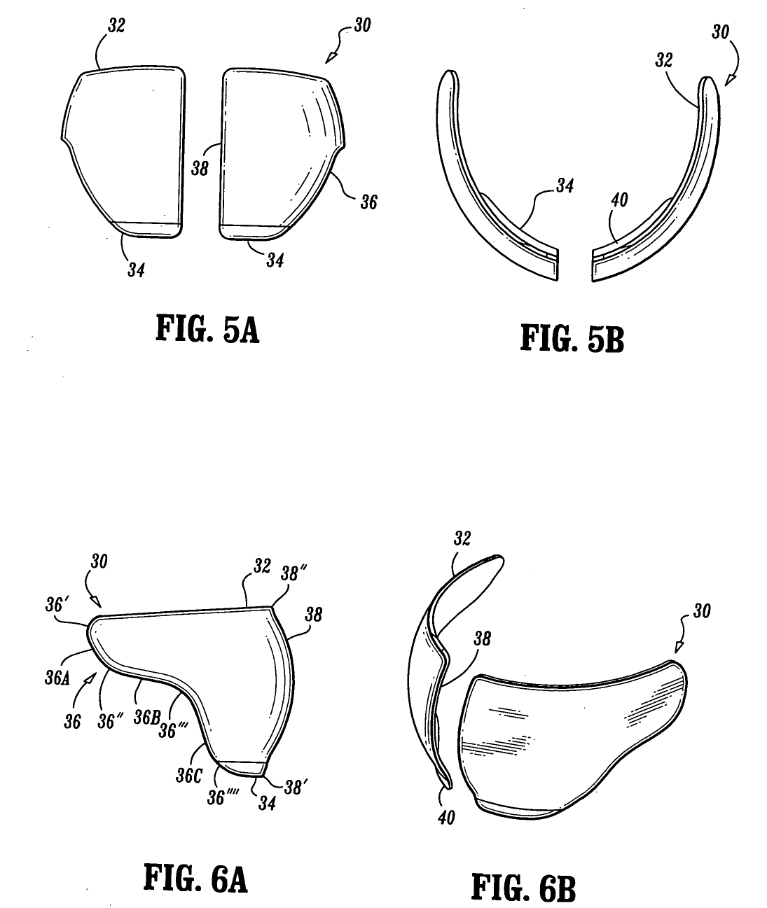

Supports 30 are shown in FIGS. 5A, 5B, 6A, and 6B. As shown in FIG. 5A, these supports are bounded by top and bottom edges 32 and 34 and two rounded side edges 36 and 38 respectively. Top and bottom edges 32 and 34 are substantially flat, while rounded side edge 36 is shaped in a curved manner so as to bend behind a user's ear. As shown in FIG. 6A, rounded side edge 36 has a first region 36a, that starts with a first curve 36′ causing first region 36a to extend down to cover a portion of a user's head above the user's ear. This first region extends to a second curve 36″, wherein edge 36 curves to a second region 36b that is shaped to extend above and back behind the user's ear. Second region 36b ends at a third curve 36″ wherein side edge 36 curves down to third region 36c that is designed to extend down to cover a substantial portion of a user's head. At this point, third region 36c reaches a fourth curve 36″″ whereby rounded side edge 36 curves to form bottom edge 34.

Bottom edge 34 extends to curve 38′ wherein at this point, rounded side edge 38 begins. Rounded side edge 38 extends up to curve 38″ whereby at this point support 30 curves to form top edge 32. Rounded side edge 38 is essentially shaped convex, so that it bows out to protect a larger portion of a user's head.

As shown in FIGS. 6A and 6B, rounded side edge 38 is designed to be curved so that support 30 can wrap around and underneath a user's head and flare out forming a protective rim 40. With this design, support 30 is essentially designed to wrap around and underneath the user's head to protect the user's head. Protective rim 40 (see also FIG. 5B) extends along the base of the user's skull so that the user's head receives protection over a substantial portion of the head.

Essentially device 10 comprising headband 20, and support pieces 30 is particularly designed to protect a user's head while still allowing sufficient size and shape adjustability for a user. For example, supports 30 are curved in region 36B to curve above a user's ears. In addition, supports 30 are also curved in region 36C to curve down and protect a base region of a user's skull. Rounded side edge 38 is also curved so that it cups around a user's head and extends underneath a base region in the user's skull via protective rim 40. While a single solid support may be used to protection an entire back region of a user's head, in a preferred embodiment, two supports 30 are used so that there is a gap or a mid region 20′ allowing headgear 10 some adjustability when securing to the user's head.

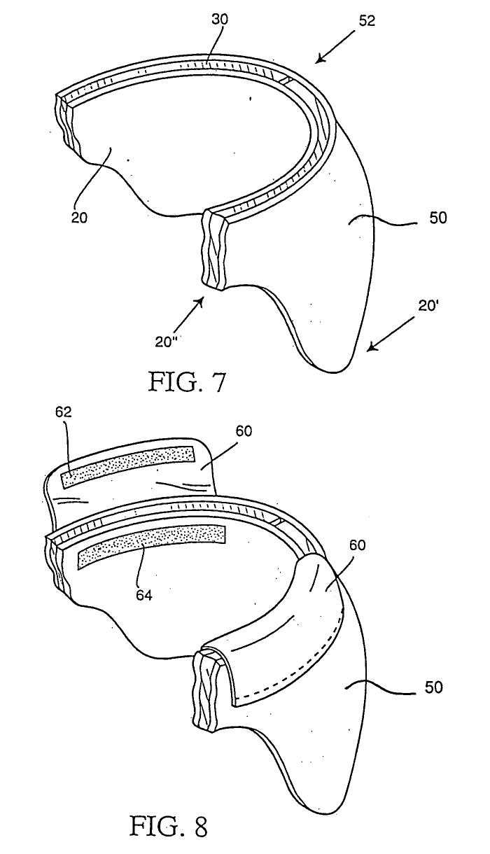

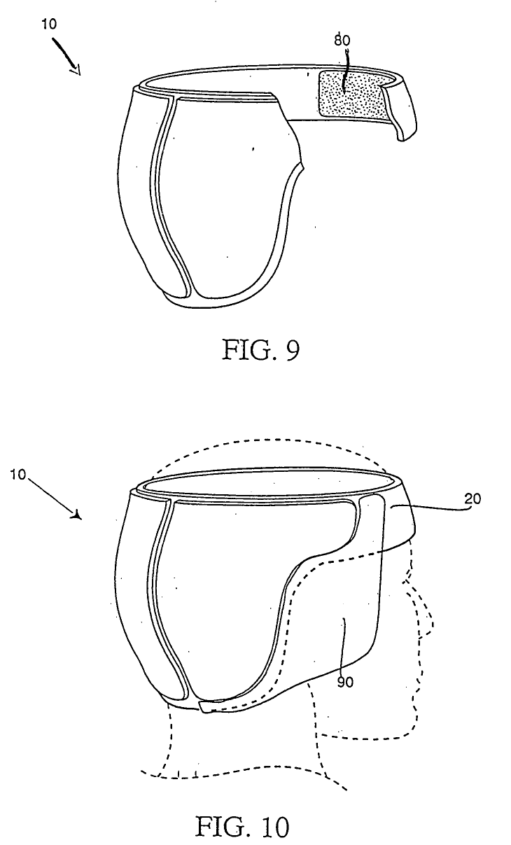

FIGS. 7 and 8 are schematic perspective views of protective headgear devices according to other exemplary embodiments of the invention. In particular, FIG. 7 depicts a protective headgear device wherein supports 30 are sandwiched between headband 20 and an additional cover 50. Headband 20 and additional cover 50 define a pocket 52 wherein pocket 52 is designed to hold supports 30 therein. Additional cover 50 is integrally attached to headband 20 at a bottom region 20′ and on a side region not shown.

In another embodiment as depicted in FIG. 8, an additional flap 60 is attached to the additional cover 50. Additional flap 60 is designed so that it has a fastener 62 that allows additional flap 60 to fold over and secure onto fastener 64 on headband 20. Essentially, additional flap 60 is divided into two pieces so that a first piece of additional flap 60 does not interfere with adjacent piece of flap 60 as it is being folded over. Fasteners 62 and 64 can combine to be a hook and loop fastener, snaps, a tongue and groove connection or any other type fastener known in the art.

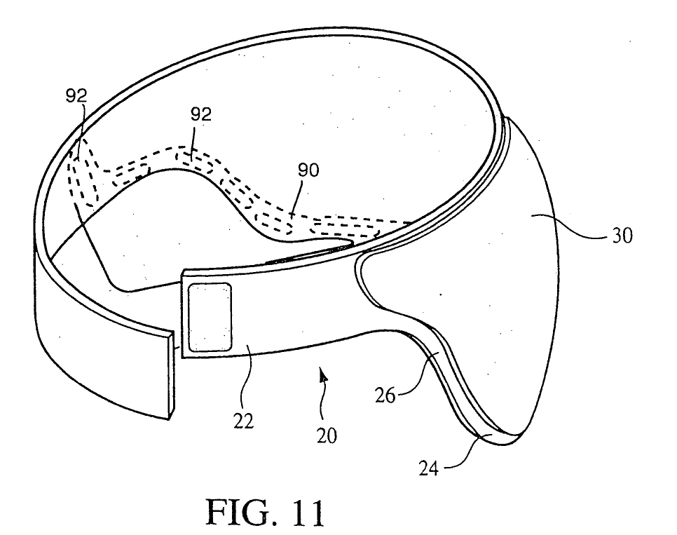

Additional components can also be added to the device. For example as shown in FIG. 9 a terry cloth piece 80 can be added to headband 20 at front region 22. Terry cloth piece 80 is designed to absorb sweat from a user and to also provide a roughened surface on headband 20 so that it remains stable on the user's forehead.

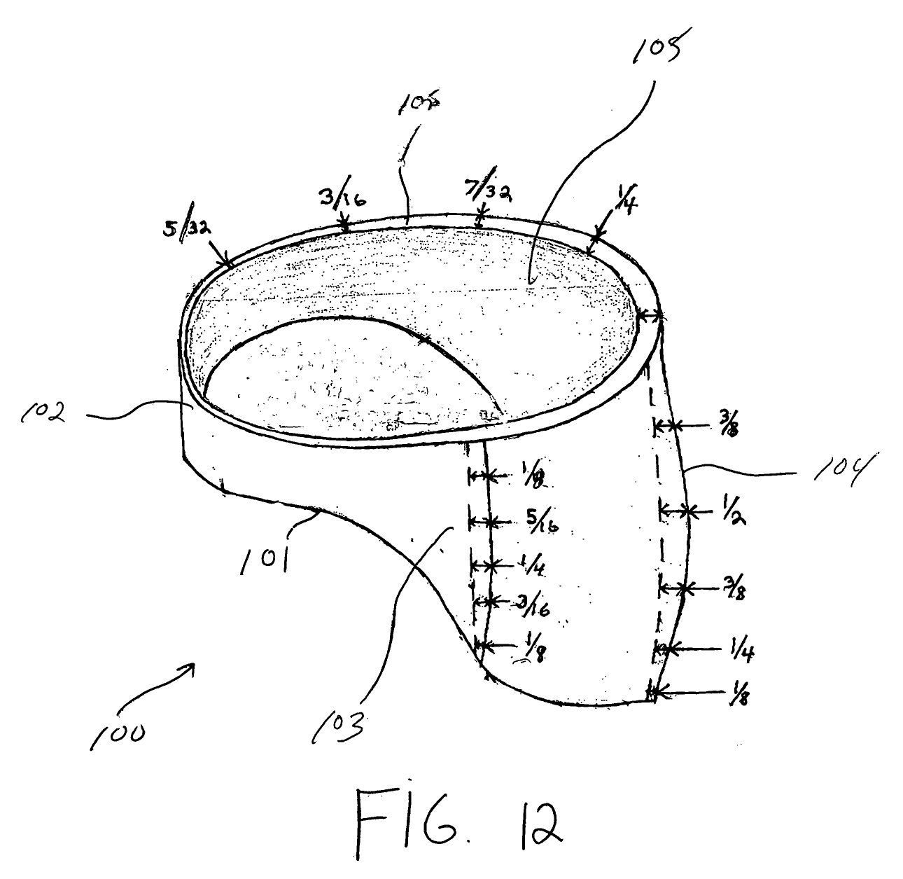

FIGS. 10 and 11 are schematic perspective views of a protective headgear device according to another exemplary embodiment of the invention. In particular, FIG. 10 depicts an exemplary headgear device similar to the devices discussed above, and further comprising additional ear pieces 90 that are designed to attach to an outside region of headband 20 and cover a user's ears. As shown in FIG. 11, additional ear piece 90 can be attached to an outside region of headband 20 via fasteners 92 extending at different angles along the ear piece. Fasteners 92 could be a hook and loop fastener, or any other type fastener known in the art. With this exemplary design, once ear piece 90 is attached, it remains secure along an outside rim of headband 20 adjacent to supports 30 because it is fixed by fasteners 92. Additional ear piece 90 is designed to provide additional warmth to a user during colder periods giving this protective headgear additional use across all seasons.

FIG. 12 is a schematic perspective view of a protective headgear device according to another exemplary embodiment of the invention. In particular, FIG. 12 illustrates a headgear device 100 comprising an integrally formed headband 101 having varying thicknesses in different regions of the headband to provide safety and comfort. The headband (20) can be formed from any suitable flexible, elastic material such as neoprene, rubber, or any other flexible cushioning material that provides impact resistance. In this exemplary embodiment, the headband 1010 comprises a front section 102, angled/tapered mid sections 103 and a back section 104. Moreover, the protective headgear device 100 comprises one or more inner lining layers 105 disposed on the inner walls of the headband 101. For example, depending on the application, the inner lining layers 105 may comprise insulative material such as fleece for warmth, or terrycloth for sweat absorption, etc, or other types of micro fiber or cushioning material for comfort.

As depicted in FIG. 12, a top band 106 increases in thickness from the front section 102 towards the back section 104. For example, as depicted, the thickness of the headband increases from 5/32″ in the front section 102 to ¼″ in the back section 104 along the upper top band section 106. Moreover, the mid sections 103 and back section 104 are formed such that the thickness of the material in such sections varies from the top band section 106 to the bottom of the mid and back sections 103 and 104. For instance, as depicted in FIG. 12, the thickness of the headband material in the mid sections 103 varies from ⅛″ to ¼″ as measured from the inner wall (dotted line) of the headband to provide increased thickness (and thus impact resistance) of the headband material in particular regions. Moreover, the thickness of the headband material in the back region 104 varies from ¼″ to ½″ as measured from the inner wall (dotted line) of the headband to provide increased thickness (and thus impact resistance) of the headband material in particular regions.

With the exemplary embodiment of FIG. 12, the integrally formed headband 101 is formed with varying thicknesses to provide increased impact resistance in various regions of the headband with increased thickness without the use of separate protective support plates. It is to be understood that the exemplary thicknesses depicted in FIG. 12 are for purposes of illustrating thickness variations of the headband 101, but the thickness variations will vary depending on the type of material used to form the headband to provide the desired impact resistance required for a given application.

In other exemplary embodiments of the invention, the integrally formed headband 101 may be formed of multiple layers of different materials to provide desired protection and ergonomic and comfort characteristics. For instance, in one exemplary embodiment of the invention, the headband 101 may comprises a flexible rubber outer shell (e.g., neoprene) and one or more inner core layers formed of foam. For instance, the foam core may comprise any suitable shock absorbing, high-impact foam material (e.g., polyethylene foam, EPS polystyrene foam, etc, which is designed to sustain multiple impacts before deterioration, or any other foam material having suitable impact-absorbing properties for a given application. In addition, the foam core may comprise an inner foam layer formed of a soft, conforming foam layer of foam for comfort.

In other exemplary embodiments of the invention, the protective headgear device 101 of FIG. 12 may be designed to enable attachment of support plates in the back region 104 for added protection, removable ear flaps, and/or other features such as described herein.

Although illustrative embodiments have been described herein with reference to the accompanying drawings, it is to be understood that the invention is not limited to the precise system and method embodiments described herein, and that various other changes and modifications may be affected therein by one skilled in the art without departing form the scope or spirit of the invention. All such changes and modifications are intended to be included within the scope of the invention as defined by the appended claims.

1. A protective headgear device, comprising:
a headband for extending around a user's head from a front region on said user's head to a back region on said user's head, and
at least one semi-rigid support coupled to said headband at a position wherein said semi-rigid support protects said back region on said user's head, wherein said at least one semi-rigid support has a curved rim region for curving around a base region of said user's head to protect said base region on said user's head.

2. The device of claim 1, wherein said headband comprises three different portions, a front portion for covering said front region of the user's head, a back portion for covering said back region of the user's head, and an expanding sloped portion for joining said front portion with said back portion, wherein said back portion is designed to cover a larger area of the user's head than said front portion.

3. The device of claim 1, wherein said headband is formed with a flexible elastic material.

4. The device of claim 1, wherein said at least one semi-rigid support is made from plastic.

5. The device of claim 1, wherein said headband comprises a releasable fastener to allow said headband to be opened and closed.

6. The device of claim 1, further comprising an absorbent insert coupled to an inside surface of said front portion of said headband.

7. The device of claim 1, further comprising a pocket for receiving said at least one semi-rigid support therein at a back portion of the headband that covers a back region of said user's head.

8. The device of claim 1, wherein a front portion of the headband that covers a front region of said user's head is narrower than a back portion of the headband that covers a back region on said user's head.

9. A protective headgear device, comprising:
an integrally formed headband that extends around a user's head from a front region on said user's head to a back region on said user's head, wherein said headband comprises a front portion for covering said front region of the user's head, a back portion for covering said back region of the user's head, and an expanding sloped portion for joining said front portion with said back portion, wherein said back portion is designed to cover a larger area of the user's head than said front portion, and wherein the headband is formed having varying thickness in different regions of the headband.
